(19) United States
(12) Patent Application Publication
(10) Pub. No.: US 2013/0172383 A1
(43) Pub. Date: Jul. 4, 2013

(54) TREATMENT OF FIBROSING DISORDERS

(71) Applicant: NOVARTIS AG, Basel (CH)

(72) Inventor: Ruey-Shiuan TSAI, Therwil (CH)

(73) Assignee: NOVARTIS AG, Basel (CH)

(21) Appl. No.: 13/773,951

(22) Filed: Feb. 22, 2013

Related U.S. Application Data
(63) Continuation of application No. 13/420,125, filed on Mar. 14, 2012, now abandoned.
Application No. 13/420,125 is a continuation of application No. 12/438,010, filed on Feb. 19, 2009, now abandoned.

(30) Foreign Application Priority Data
Aug. 22, 2006 (EP) 06119311.6
Oct. 6, 2006 (EP) 06121903.6

(51) Int. Cl. A61K 31/439; A61K 45/06

(52) U.S. Cl. CPC A61K 31/439; A61K 45/06
USPC 514/291

(57) ABSTRACT

A method for treating liver-associated fibrosing disorders or lupus, comprising administering to a subject in need thereof a therapeutically effective amount of a compound of formula

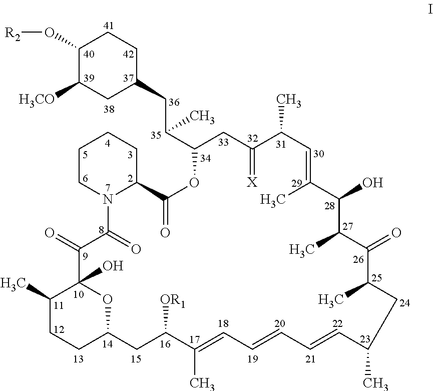

optionally in conjunction with a co-agent.

The present invention relates to the treatment of liver-associated fibrosing disorders and lupus, which is a kidney-associated fibrosing disorder, more specifically to the use of a compound of formula I, as specified herein, for the treatment of liver-associated fibrosing disorders or lupus.

Fibrosis or fibroplasia, in which connective tissue replaces normal parenchymal tissue, is a pathological process that results from improper repairs during tissue or organ injuries caused by infections, autoimmune reactions, chemical intoxication or mechanical assaults.

Fibrosing disorders can occur in main organs or tissues, such as the liver. Lupus nephritis is a kidney-associated fibrosing disorder, namely an inflammation of the kidney caused by systemic lupus erythematosus (SLE). Liver-associated fibrosis; such as hepatic fibrosis and cirrhosis can take place following chronic injury caused by various etiologies. Various insults on the liver, including infection (e.g. hepatitis B virus, hepatitis C virus), alcohol, autoimmune diseases or genetic abnormalities, can lead the hepatic cells to scar tissue production (fibrosis) or to severe fibrotic changes and a breakdown in the normal architecture of the liver (cirrhosis). The hepatic fibrosis or cirrhosis may eventually result in complete liver failure with the need for a liver transplant.

The liver-associated fibrosing disorders include for example infection-induced liver fibrosis or cirrhosis, such as post hepatitis C or post hepatitis B cirrhosis (hepatic fibrosis), drug-induced liver fibrosis or cirrhosis, chemical-induced liver fibrosis or cirrhosis (e.g. alcohol cirrhosis), autoimmune-induced liver fibrosis or cirrhosis, genetic hemochromatosis.

Disorders as used herein include diseases.

Rapamycin is a known macrolide antibiotic produced by Streptomyces hygroscopicus. Compounds which are useful according to the present invention include a compound of formula

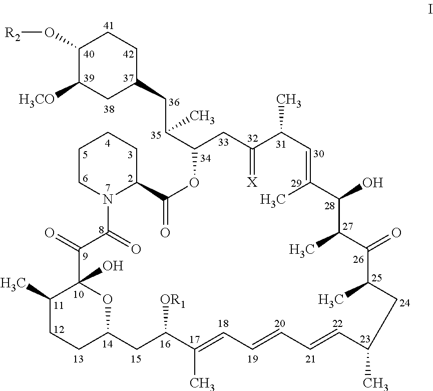

wherein
R₁ is CH₃ or C₃₋₆alkynyl,

### R₂ is H, —CH₂-CH₂-OH, —CH₂-CH₂-O-CH₂-CH₃,

### X is =O, (H, H) or (H, OH),

provided that R₂ is other than H when X is ═O and R₁ is CH₃.

In a compound of formula I each single substituent indicated may be a preferred substituent, independently of any other substituent defined.

Representative examples of compounds of formula I include e.g. 40-O-(2-hydroxy)-ethyl-rapamycin, 32-deoxorapamycin, 16-pent-2-ynyloxy-32-deoxorapamycin, 16-pent-2-ynyloxy-32 (S or R)-dihydro-rapamycin, 16-pent-2-ynyloxy-32 (S or R)-dihydro-40-O-(2-hydroxy)-ethyl-rapamycin, and 40-O-(2-ethoxy)-ethyl-rapamycin, such as
40-O-(2-hydroxy)-ethyl-rapamycin, and/or
32-deoxorapamycin, and/or
16-pent-2-ynyloxy-32-deoxorapamycin, and/or
16-pent-2-ynyloxy-32 (S or R)-dihydro-rapamycin, and/or
16-pent-2-ynyloxy-32 (S or R)-dihydro-40-O-(2-hydroxy)-ethyl-rapamycin, and/or
40-O-(2-ethoxy)-ethyl-rapamycin.

Preferably a compound of formula I is 40-O-(2-hydroxy)-ethyl-rapamycin (everolimus).

According to the present invention it was surprisingly found that compounds of formula I are useful for the treatment of liver-associated fibrosing disorders and lupus, e.g. compounds of formula I may inhibit or decrease fibrotic processes, e.g. through one or more of the following mechanisms:
inhibition of epithelial to mesenchymal transition,
reduction of expression of profibrotic growth factors,
reduction of extracellular matrix production.

In accordance with the particular findings the present invention provides in several aspects:

1.1 A method for treating liver-associated fibrosing disorders or lupus, comprising administering to a subject in need thereof a therapeutically effective amount of a compound of formula I.

Lupus as used herein includes lupus nephritis and (systemic) lupus erythematosus (SLE), preferably lupus nephritis.

1.2 A method for inhibiting epithelial to mesenchymal transition, comprising administering to a subject in need thereof a therapeutically effective amount of a compound of formula I.

1.3 A method for reduction of expression of profibrotic growth factors, comprising administering to a subject in need thereof a therapeutically effective amount of a compound of formula I.

1.4 A method for reduction of extracellular matrix production, comprising administering to a subject in need thereof a therapeutically effective amount of a compound of formula I.

1.5 A method for treating liver fibrosis, comprising administering to a subject in need thereof a therapeutically effective amount of a compound of formula I.

1.6 A method for treating liver cirrhosis, comprising administering to a subject in need thereof a therapeutically effective amount of a compound of formula I.

1.7 A method for treating lupus, comprising administering to a subject in need thereof a therapeutically effective amount of a compound of formula I.

1.8 A method for treating lupus nephritis, comprising administering to a subject in need thereof a therapeutically effective amount of a compound of formula I.

In a further aspect the present invention also provides

1.9 A method for the treatment of a disease associated with any disease condition as indicated in 1.1 to 1.8 above, comprising administering to a subject in need thereof a therapeutically effective amount of a compound of formula I.

Treatment as used herein includes treatment or prevention, preferably treatment.

In another aspect the present invention provides

1.10 A method as indicated under 1.1 to 1.9 above, wherein a compound of formula I is selected from, e.g. selected from the group consisting of, 40-O-(2-hydroxy)-ethyl-rapamycin, 32-deoxorapamycin, 16-pent-2-ynyloxy-32-deoxorapamycin, 16-pent-2-ynyloxy-32 (S or R)-dihydro-rapamycin, 16-pent-2-ynyloxy-32 (S or R)-dihydro-40-O-(2-hydroxy)-ethyl-rapamycin and 40-O-(2-ethoxy)-ethyl-rapamycin,

such as 40-O-(2-hydroxy)-ethyl-rapamycin (everolimus).

In other aspects the present invention provides:

2. A compound of formula I for use in any method as defined under 1.1 to 1.10 above.

3. A compound of formula I for the preparation of a medicament, e.g. a pharmaceutical composition, for use in any method as defined under 1.1 to 1.10 above.

4. A pharmaceutical composition for use in any method as defined under 1.1 to 1.10 above, comprising a compound of formula I together with one or more pharmaceutically acceptable diluents or carriers therefore.

A compound of formula I may be used in a method or for a use provided by the present invention as the sole active ingredient (agent), or in conjunction with a second drug substance which is a chemotherapeutic agent.

By the term “chemotherapeutic agent” is meant especially any chemotherapeutic agent other than a compound of formula I which provides a benefit in combined treatment compared with single treatment, e.g. in a method or for a use provided by the present invention. Preferably such chemotherapeutic agent is an agent which provides a beneficial effect in the treatment of fibrosis, such as an antifibrotic agent, e.g. including an agent which provides a synergestic effect in combined treatment with a compound of formula I.

Appropriate antifibrotic agents e.g. include
inhibitors of the renin-angiotensin system, such as renin inhibitors, e.g. including aliskiren, SPP630, SPP635, SPP800, Ro 42-5892; angiotensin receptor antagonists, such as losartan, valsartan, irbesartan, eprosartan, candesartan, olmesartan (medoxomil), telmisartan; angiotensin converting enzyme (ACE) inhibitors, such as benazepril, captopril enalapril, fosinopril, lisinopril, moexipril, perindopril, quinapril, ramipril, trandolapril;
connective tissue growth factor (CTGF) antagonists, such as antibodies against connective tissue growth factor, or statins, such as atorvastatin, simvastatin, cerivastatin, pitavastatin, fluvastatin, lovastatin, pravastatin, rosuvastatin;
platelet-derived growth factor (PDGF) antagonists, such as Trapidil®, antibodies against platelet-derived growth factor, PDGF receptor tyrosine kinase inhibitors, e.g. SU9518, imatinib, sunitinib (malate), AMN107, BMS354825;
fibroblast growth factor (FGF) antagonists, e.g. antibodies against fibroblast growth factor, FGF receptor tyrosine kinase inhibitors, e.g. suramine (sodium),
tumor necrosis factor alpha (TNF-alpha) antagonists, such asTNF-alpha antibodies, e.g. infliximab, TNF-alpha receptor Ig constructs,
interferon gamma, relaxin,
endothelin receptor antagonists, e.g. BQ-123, bosentan, clazosentan, SPP301;
transforming growth factor beta (TGF-beta) antagonists, e.g. batimastat, or TGF-beta antibodies, activin receptor-like kinase inhibitors, such as SB-431542,
vascular endothelial growth factor (VEGF) antagonists, e.g. or VEGF antibodies; such as bevacizumab, ranibizumab; VEGF receptor tyrosine kinase inhibitors, e.g. PTK787/ZK 222584, ZD6474, SU5416, ABT-869, AEE788;
interleukin 13 antagonist, interleukin 33 antagonist.

In another aspect the present invention provides:

5.1 A pharmaceutical combination, e.g. pharmaceutical composition, e.g. for use as defined under 1.1 to 1.10 above, comprising

a) a first agent which is a compound of formula I, and
b) a second drug substance as a co-agent which is a chemotherapeutic agent, e.g. an antifibrotic agent, such as defined herein.

Pharmaceutical combinations include fixed combinations, in which two or more pharmaceutically active agents, such as a compound of formula I and a chemotherapeutic agent, are in the same formulation; kits, in which two or more pharmaceutically active agents, such as a compound of formula I and a chemotherapeutic agent, in separate formulations are sold in the same package, e.g. with instruction for co-administration; and free combinations in which the pharmaceutically active agents, such as a compound of formula I and a chemotherapeutic agent, are packaged separately, but instruction for concomitant or in sequential administration are given.

In another aspect the present invention provides:

5.2 A pharmaceutical package comprising a first drug substance which is a compound of formula I, and at least one second drug substance, said second drug substance being a chemotherapeutic agent, e.g. as defined herein, beside instructions for combined administration;

5.3 A pharmaceutical package comprising a first drug substance which is a compound of formula I, beside instructions for combined administration with at least one second drug substance, said second drug substance being a chemotherapeutic agent, e.g. as defined herein;

5.4 A pharmaceutical package comprising at least one chemotherapeutic agent, e.g. as defined herein, beside instructions for combined administration with a compound of formula I;

e.g, for any use or in any method as provided by the present invention.

6. Any method as defined above comprising co-administrating, e.g. concomitantly or in sequence, a therapeutically effective amount of a compound of formula I and a second drug substance, said second drug substance being a chemotherapeutic agent, e.g. as defined herein.

Treatment with combinations according to the present invention may provide improvements, e.g. benefits, compared with single treatment (mono-therapy).

In another aspect the present invention provides
A pharmaceutical combination comprising an amount of a compound of formula I and an amount of a chemotherapeutic agent, e.g. such as defined herein, wherein the amounts are appropriate to produce a beneficial effect compared with single treatment, e.g. compared with mono-therapy, such as a synergistic therapeutic effect;
A method for improving the therapeutic utility of a compound of formula I, comprising co-administrating, e.g. concomitantly or in sequence, a therapeutically effective amount of a compound of formula I and a chemotherapeutic agent, e.g. such as defined herein;
A method for improving the therapeutic utility of a chemotherapeutic agent, e.g. such as defined herein, comprising co-administrating, e.g. concomitantly or in sequence, a compound of formula I and a chemotherapeutic agent, e.g. such as defined herein;
   e.g. for use in any method or for any use as provided by the present invention.

Treatment includes treatment and prevention (prophylaxis).

For such treatment, the appropriate dosage will, of course, vary depending upon, for example, the chemical nature and the pharmacokinetic data of the active ingredient, such as a compound of formula I, and/or the chemotherapeutic agent, the individual host, the mode of administration and the nature and severity of the conditions being treated. However, in general, for satisfactory results in larger mammals, for example humans, an indicated daily dosage includes a range
from about 0.0001 g to about 1.5 g, such as 0.0001 g to 1.5 g;
from about 0.01 mg/kg body weight to about 20 mg/kg body weight, such as 0.01 mg/kg body weight to 20 mg/kg body weight, for example administered in divided doses up to four times a day.

For example, everolimus may be administered in dosages from (about) 0.1 mg up to (about) 15 mg, such as 0.1 mg to 10 mg, e.g. 0.1 mg, 0.25 mg, 0.5 mg, 0.75 mg, 1.0 mg, 2.5 mg, 5 mg, 10 mg, e.g. in a weekly dosage of (about) 1 mg up to 70 mg.

Other compounds of formula I may be used as appropriate, e.g. in similar dosages as indicated for everolimus.

Chemotherapeutic agents, as described herein, may be used in dosages as appropriate, e.g. according, e.g. analogously, as described for their administration in mono-therapy, e.g. in case of synergism with a compound of formula I, even below such dosages.

A compound of formula I, or a chemotherapeutic agent as described herein may be administered by any conventional route, for example enterally, e.g. including nasal, buccal, rectal, oral, administration; parenterally, e.g. including intravenous, intraarterial, intramuscular, intracardiac, subcutanous, intraosseous infusion, transdermal (diffusion through the intact skin), transmucosal (diffusion through a mucous membrane), inhalational administration; topically; e.g. including intranasal, intratracheal administration; intraperitoneal (infusion or injection into the peritoneal cavity); epidural (peridural) (injection or infusion into the epidural space); intrathecal (injection or infusion into the cerebrospinal fluid); intravitreal (administration via the eye) administration; or via medical devices, e.g. for local delivery, e.g. stents;

e.g. in form of coated or uncoated tablets, capsules, (injectable) solutions, infusion solutions, solid solutions, suspensions, dispersions, solid dispersions; e.g. in the form of ampoules, vials, in the form of inhaler powder, foams, in the form of suppositories;

In each case where active agents are indicated herein, such as a compound of formula I, or another chemotherapeutic agent, e.g. as indicated herein, any compound indicated comprises the compound, pharmaceutical acceptable salts thereof, corresponding isomeric forms, such as racemates, diastereoisomers, enantiomers, tautomers, e.g. in pure form or in form of isomeric mixtures, as well as corresponding crystal modifications, e.g. solvates, hydrates and polymorphs. The compounds used as active ingredients in the combinations of the invention may be prepared and administered as described in their product description, respectively. Also within the scope of this invention is the combination of more than two separate active ingredients as set forth above, namely a pharmaceutical combination within the scope of this invention could include three active ingredients or more. Further, both the first agent and the co-agent are not the identical ingredient.

Pharmaceutical compositions according to the present invention may be manufactured according, e.g. analogously, to a method as conventional, e.g. by mixing, granulating, coating, dissolving or lyophilizing processes. Unit dosage forms may contain, for example, from about 0.1 mg to about 1500 mg, such as 1 mg to about 1000 mg.

Pharmaceutical compositions indicated herein, comprising a compound of formula I, a chemotherapeutic agent, e.g. as described herein, or a combination according to (provided by) the present invention may be provided as appropriate, e.g. according, e.g. analogously, to a method as conventional, or as indicated herein.

Appropriate in vitro and in vivo models and assays for liver associated fibrosing disorders, such as hepatic fibrosis and hepatic cirrhosis, are known or may be provided as appropriate, see e.g. J. Zhu et al, Gastroenterology, November 1999, 117(5):, p. 1198-1204, N. Shibata et al, Cell transplant, 2003, 12(5), p. 499-507.

Appropriate in vivo models and assays for lupus are known or may be provided as appropriate, see e.g. J Gavalchin et al, The Journal of Immunology, Vol 138, 1987, Issue 1, pages 128-137 and 138-148, Alan D. Salama, Drug Discovery Today: Disease Models, Volume 1, Issue 4, December 2004, p. 457-463, ML Stoll, J Gavalchin, Rheumatology (Oxford), January 2000, Volume 39, Issue 1, p. 18-27.

Compounds of formula I, optionally in combination with one or more chemotherapeutic agent, e.g. such as disclosed herein, show activity in such models/assays.

1. A method for treating liver-associated fibrosing disorders or lupus, comprising administering to a subject in need thereof a therapeutically effective amount of a compound of a compound of formula 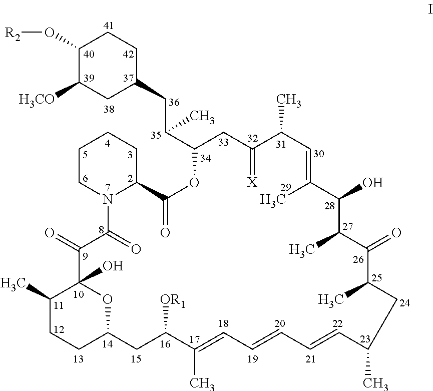
wherein
R₁ is CH₃ or C₃₋₆alkynyl,
R₂ is H, —CH₂-CH₂-OH, —CH₂-CH₂-O-CH₂-CH₃,
X is ═O, (H, H) or (H, OH),
provided that R₂ is other than H when X is ═O and R₁ is CH₃.

2. A method for inhibiting epithelial to mesenchymal transition, comprising administering to a subject in need thereof a therapeutically effective amount of a compound of formula I.

3. A method for reduction of expression of profibrotic growth factors, comprising administering to a subject in need thereof a therapeutically effective amount of a compound of formula I.

4. A method for reduction of extracellular matrix production, comprising administering to a subject in need thereof a therapeutically effective amount of a compound of formula I.

5. The method according to claim 1 for treating liver fibrosis.

6. The method according to claim 5 for treating liver cirrhosis.

7. The method according to claim 1 for treating lupus.

8. The method according to claim 7 for treating lupus nephritis.

9. A method for the treatment of a disease associated with any disease condition as indicated in claim 1, comprising administering to a subject in need thereof a therapeutically effective amount of a compound of formula I as defined in claim 1.

10. The method according to claim 1, wherein the compound of formula I is selected from the group consisting of 40-O-(2-hydroxy)-ethyl-rapamycin, 32-deoxorapamycin, 16-pent-2-ynyloxy-32-deoxorapamycin, 16-pent-2-ynyloxy-32 (S or R)-dihydro-rapamycin, 16-pent-2-ynyloxy-32 (S or R)-dihydro-40-O-(2-hydroxy)-ethyl-rapamycin, and 40-O-(2-ethoxy)-ethyl-rapamycin.

11. The method according to claim 1, wherein the compound of formula I is 40-O-(2-hydroxy)-ethyl-rapamycin.

12.-15. (canceled)

16. The method according to claim 1, wherein said compound of formula I is administered as part of a pharmaceutical composition comprising one or more pharmaceutically acceptable diluents or carriers therefore.

17. The method according to claim 16, wherein said pharmaceutical composition further comprises a second drug substance as a co-agent which is a chemotherapeutic agent.
